(19) United States
(12) Patent Application Publication
(10) Pub. No.: US 2024/0325402 A1
(43) Pub. Date: Oct. 3, 2024

(54) MOMELOTINIB COMBINATION THERAPY

(71) Applicant: GlaxoSmithKline LLC, Wilmington, DE (US)

(72) Inventor: Bryan William STROUSE, Wilmington, DE (US)

(21) Appl. No.: 18/293,935

(22) PCT Filed: Aug. 8, 2022
(86) PCT No.: PCT/US2022/074645

§ 371 Date: Jan. 31, 2024

Related U.S. Application Data
(60) Provisional application No. 63/231,432, filed on Aug. 10, 2021.

(51) Int. Cl. A61K 31/5377; A61K 45/06

(52) U.S. Cl. CPC A61K 31/5377; A61K 45/06

(57) ABSTRACT

The present disclosure relates to methods of treating an inflammatory disease or disorder, the method comprising: administering to a subject in need thereof a therapeutically effective amount of momelotinib (MMB) or a pharmaceutically acceptable salt thereof; and administering to the subject one or more anti-inflammatory agents, and combinations for use in treating an inflammatory disease or disorder.

### 1. INTRODUCTION

Myelofibrosis (MF) is a progressive myeloproliferative neoplasm driven by constitutive activation of the JAK-STAT pathway. JAK-STAT activation can lead to progressive bone marrow fibrosis, triggering local and systemic inflammation, ultimately resulting in three disease hallmarks: anemia, constitutional symptoms and splenomegaly.

Additionally, there is evidence of STAT activation in malignant tumors, among them lung, breast, colon, ovarian, prostate and liver cancer, as well as Hodgkin's lymphoma, multiple myeloma and hepatocellular carcinoma. Chromosomal translocations involving JAK2 fusions to Tel, Ber and PCMI have been described in a number of hematopoietic malignancies including chronic myelogenous leukemia (CML), acute myelogenous leukemia (AML), chronic eosinophilic leukemia (CEL), myclodysplastic syndrome (MDS), myeloproliferative disease (MPD) and acute lymphocytic leukemia (ALL). This suggests treatment of hyperproliferative disorders such as cancers including multiple myeloma; prostate, breast and lung cancer; Hodgkin's Lymphoma; CML; AML; CEL; MDS; ALL; B-cell Chronic Lymphocytic Leukemia; metastatic melanoma; glioma; and hepatoma, by JAK inhibitors is indicated.

The bromodomain and extraterminal (BET) family of proteins present a transcriptional vulnerability in human cancer. Small molecules targeting BET proteins suppress the transcription of cancer-promoting genes elicit anticancer activity in numerous malignant contexts. Kinase inhibitors, including JAK2 inhibitors, may also inhibit BET bromodomains (Martin et al. ACS Chem Biol. 2013; 8(11):2360-5; Ciceri et al. Nat Chem Biol. 2014; 10(4):305-12; Dittmann et al. ACS Chem Biol. 2014; 9(2):495-502; Ember et al. ACS Chem Biol. 2014; 9(5):1160-71). The effect of JAK2 inhibitors on BET proteins may contribute to the effectiveness of the inhibitors.

Small molecule inhibitors of the JAK-STAT pathway and ACVR1/SMAD signaling may be useful in treatment of kinase associated diseases such as immunological and inflammatory diseases including organ transplants; hyperproliferative diseases including cancer and myeloproliferative diseases; viral diseases; metabolic diseases; and vascular diseases.

Small molecule inhibitors of the JAK-STAT pathway have been developed in MF, including two currently approved therapies: ruxolitinib (JAK1 and JAK2) and fedratinib (JAK2). Although these agents can provide benefit for splenomegaly and constitutional symptoms, clinical utility can be limited by their myelosuppressive properties. Further, JAK inhibitors are not curative in MF and consequently there is increasing interest in identifying efficacious JAK inhibitor combination therapies.

Momelotinib (MMB) is a potent nanomolar inhibitor of JAK1, JAK2 and uniquely among the JAKi class, ACVR1. This differentiated profile results in a suite of anemia benefits, including restoration of iron homeostasis and red blood cell production and reduction or elimination of RBC transfusions. MMB has also shown to improve or maintains platelet counts. This reduced myelosuppression and favorable safety profile enables sustained, near-maximal dose intensity which is accompanied by clinically comparable spleen and symptom benefits to ruxolitinib.

### 2. SUMMARY OF THE INVENTION

Disclosed herein are methods of treating an inflammatory disease or disorder in a subject comprising administering to a subject in need thereof a therapeutically effective amount of momelotinib (MMB) or a pharmaceutically acceptable salt thereof, and administering to the subject one or more additional anti-inflammatory agents.

Disclosed herein are methods of treating cancer in a subject comprising administering to a subject in need thereof a therapeutically effective amount of momelotinib (MMB) or a pharmaceutically acceptable salt thereof, and administering to the subject one or more additional anti-inflammatory agents.

In some embodiments, the one or more additional anti-inflammatory agents are agents that modulate NF-κB activity. An agent that modulates NF-κB activity can be an inhibitor of the NF-κB pathway in a cell. An agent that modulates NF-κB activity can be an antagonist of NF-κB.

In some embodiments, the one or more anti-inflammatory agents is a BET protein inhibitor, for example a BRD4 inhibitor. In some embodiments, the BET protein inhibitor is selected from GSK2820151, GSK525762, GS-5829, RO6870810 (IV), BAY1238097, CC-90010, BMS-986158, INCB054329, INCB057643, ODM-207, AZD5153, FT-1101, ABBV-744, ABBV-075, PLX51107, BI894999, OTX015/MK8628, ZEN003694, RVX-000222, CPI-0610, apabetalone and fedratinib. In some embodiments, the BET protein inhibitor is CPI-0610.

In some embodiments, the one or more anti-inflammatory agents is an IKK protein inhibitor, for example, an IKKα, IKKβ, and/or IKKε inhibitor. In some embodiments, the IKK inhibitor is selected from B1605906, MLN120B, PHA-408, LY2409881, PS-1145, and BMS-345541.

In some embodiments, the one or more anti-inflammatory agents is an IRAK and/or TLR inhibitor, for example, an IRAK1 inhibitor or IRAK4 inhibitor. In some embodiments, the IRAK and/or TLR inhibitor is selected from BAY1 834845, CA-4948, PF-06650833 and pacritinib.

In some embodiments, the one or more anti-inflammatory agents is a protein or small molecule agent.

In some embodiments, methods for treating the inflammatory disease or disorder comprises at least ameliorating one or more symptoms of the disease or disorder, for example, wherein amelioration of the one or more symptoms is increased as compared to a monotherapy with momelotinib (MMB) or a monotherapy with the one or more anti-inflammatory agents alone.

In some embodiments, the method of treatment results in reduction in NF-KB pathway activity and modulation of JAK-STAT and ACVR1/SMAD signaling in cells of the subject.

In some embodiments of the disclosure, the subject is a mammal. In some embodiments, the subject is human. In some embodiments, the subject has a chronic inflammatory disease, an autoimmune disease or cancer. In some embodiments the subject has a chronic inflammatory disease. In some embodiments, the subject has cancer. In some embodiments, the subject has myelofibrosis.

Disclosed herein are methods of treating myelofibrosis in a subject comprising administering to a subject in need thereof a therapeutically effective amount of momelotinib (MMB) or a pharmaceutically acceptable salt thereof, and administering to the subject CPI-0610.

In some embodiments, the MMB or a pharmaceutically acceptable salt thereof is administered orally. In some embodiments, the MMB or a pharmaceutically acceptable salt thereof is administered daily or weekly. In some embodiments, the MMB or a pharmaceutically acceptable salt thereof is administered intermittently.

In some embodiment, the present disclosure relates to a combination of a therapeutically effective amount of momelotinib (MMB) or a pharmaceutically acceptable salt thereof, and one or more additional anti-inflammatory agents for use in the methods of the present disclosure. i.e. the present disclosure includes a combination of a therapeutically effective amount of momelotinib (MMB) or a pharmaceutically acceptable salt thereof, and one or more additional anti-inflammatory agents for use the treatment of an inflammatory disease or disorder in a subject. In some embodiments, the present disclosure includes the use of a combination of a therapeutically effective amount of momelotinib (MMB) or a pharmaceutically acceptable salt thereof, and one or more additional anti-inflammatory agents in the manufacture of a medicament for the treatment of an inflammatory disease or disorder in a subject.

### BRIEF DESCRIPTION OF THE DRAWING

FIG. 1 shows the overlay graph demonstrating the effects of the tested compounds on signaling pathway reporter activity.

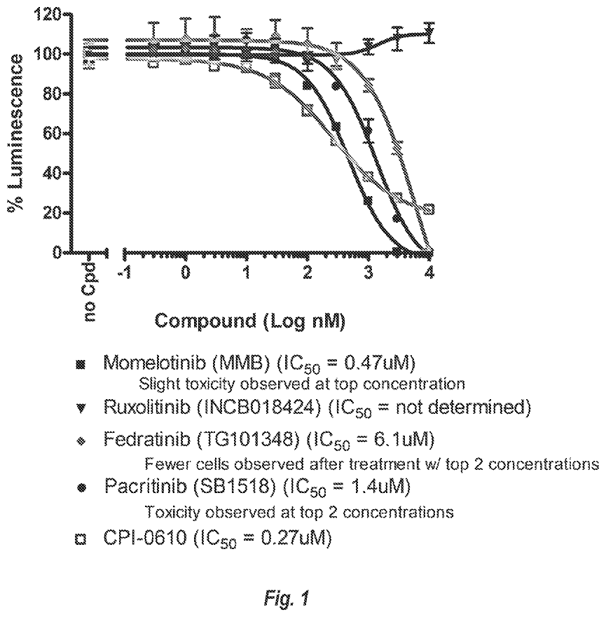

### 3. DESCRIPTION

Terms used in the claims and specification are defined as set forth below unless otherwise specified.

The term “ameliorating” refers to any therapeutically beneficial result in the treatment of a disease state, e.g., an arthritic disease state, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term “subject” broadly refers to any animal, including but not limited to, human and non-human animals (e.g., dogs, cats, cows, horses, sheep, pigs, poultry, fish, crustaceans, etc.).

The term “effective amount” refers to the amount of a composition sufficient to effect beneficial or desired results. An effective amount can be administered in one or more administrations, applications or dosages and is not intended to be limited to a particular formulation or administration route.

The term “therapeutically effective amount” is an amount that is effective to ameliorate a symptom of a disease. A therapeutically effective amount can be a “prophylactically effective amount” as prophylaxis can be considered therapy.

The terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

The term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

The terms “pharmaceutically acceptable” or “pharmacologically acceptable,” as used herein, refer to compositions that do not substantially produce adverse reactions, e.g., toxic, allergic, or immunological reactions, when administered to a subject.

It must be noted that, as used in the specification and the appended claims, the singular forms “a,” “an” and “the” include plural referents unless the context clearly dictates otherwise.

In some embodiments, the subjects treated by methods disclosed herein have a chronic inflammatory disease, an autoimmune disease, or a hyperproliferative disease. In some embodiments, the subjects treated by methods disclosed herein have cancer and/or myeloproliferative disease. Nonlimiting examples of cancer and myeloproliferative diseases include cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adeno carcinoma), alveolar (bronchiolar) carcinoma, bronchial adenoma, sarcoma, lymphoma, chondromatous hanlartoma, inesothelioma: Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), Small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, Villous adenoma, hamartoma, leiomyoma); Genitourinary tract: kidney (adenocarcinoma, Wilm's tumor nephroblastoma, lymphoma, leukemia), bladder and urethra (squamous cell carcinoma, transitional cell carcinoma, adenocarcinoma), prostrate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfronna (osteocartilaginous exostoses), benign chondroma, chondroblastoma, chondromyxofibroma, osteoid osteoma and giant cell tumors; Nervous system: skull (osteoma, hemangioma, granuloma, Xanthoma, osteitis deformians), meninges (meningioma, meningiosarcoma, gliomatosis), brain (astrocytoma, medulloblastoma, glioma, ependymoma, germinoma pinealoma, glioblastoma multiform, oligodendroglioma, Schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma serous cystadenocarcinoma, mucinous cystadenocarcinoma, unclassified carcinoma, granulosa-thecal cell tumors, SertoliLeydig cell tumors, dysgerminoma, malignant teratoma), Vulva (squamous cell carcinoma, intraepithelial carcinoma, adenocarcinoma, fibrosarcoma, melanoma), vagina (clear cell carcinoma, squamous cell carcinoma, botryoid sarcomaembryonal rhabdomyosarcoma), fallopian tubes (carcinoma); Hematologic: blood (myeloid leukemia acute and chronic, acute lymphoblastic leukemia, chronic lymphocytic leukemia, multiple myeloma, myelodysplastic syndrome), Hodgkin's disease, non-Hodgkin's lymphoma malignant lymphoma; Skin: malignant melanoma, basal cell carcinoma, squamous cell carcinoma, Karposi's sarcoma, moles dysplastic nevi, lipoma, angioma, dermatofibroma, keloids, psoriasis; Adrenal glands: neuroblastoma; and Myeloproliferative diseases such as polycythemia Vera (PV), primary myelofibrosis (MF), thrombocythemia, essential thrombocythemia (ET), agnoneic myeloid metaplasia (AMM), also referred to asidiopathic myelofibrosis (IMF), chronic myelogenous leuke mia (CML), Systemic mastocystosis (SM), chronic neutrophilic leukemia (CNL), myelodisplastic syndrome (MDS) and systemic mast cell disease (SMCD).

NF-κB (nuclear factor kappa-light-chain-enhancer of activated B cells) is a protein complex that controls transcription of DNA, cytokine production and cell survival. NF-κB plays a role in regulating the immune response to infection. Incorrect regulation of NF-κB has been linked to e.g., cancer, inflammatory and autoimmune diseases.

The transcriptional activity of NF-κB is regulated by several distinct pathways. One is the canonical pathway, which is induced by pro-inflammatory cytokines. Engagement of the canonical NF-κB pathway triggers signaling cascades that converge on activation of the IκB kinase (IKK) complex, which is formed by the kinase subunits, IKKα and IKKβ, and a regulatory subunit IKKγ (also known as NEMO, NF-κB essential modifier).

Interleukin-I receptor-associated kinases (IRAK1, IRAK2, IRAK3 [IRAK-M], and IRAK4) are serine-threonine kinases involved in the toll-like receptor (TLR) and interleukin-1 signaling pathways, through which they regulate innate immunity and inflammation.

Inhibition of BET (Bromodomain and Extra-Terminal domain) protein family members such as BRD4, among others, is implicated in NF-κB-dependent promoter and super-enhancer modulation.

The efficacy of BET inhibitors has also been shown in preclinical solid tumor models, including tumors of the prostate, breast, colon, intestine, pancreas, liver, and brain (Sahai et al. Oncotarget. 2016; 7(33):53997-54009). In some instances specific subsets of tumors harbor exceptional sensitivity to BET inhibitors (Rathert et al. Nature. 2015; 525(7570):543-547).

### Anti-Inflammatory Agents

Any of the anti-inflammatory agents described herein can be utilized in the subject method in conjunction with momelotinib. In some embodiments, the anti-inflammatory agent is an NF-κB modulating agent, an agent that modulates an activity or function of the NF-κB regulatory pathway. A NF-κB modulating agent can be an agent that interacts directly with the NF-κB protein complex, or an agent that indirectly modulates the activity of NF-κB in a cell, e.g., by inhibiting the activity of a kinase target in a NF-κB regulatory pathway (e.g., as described herein).

In some embodiments, a NF-kB modulating agent of interest has anti-NFKB activity that is a consequence of inhibitory activity on an IRAK target (e.g., IRAK1), an IKK target (e.g., IKK-β), and/or a BET protein (e.g., BRD4).

In some embodiments, the anti-inflammatory agents is a BET protein inhibitor. In some embodiments, the BET protein inhibitor is a BRD4 inhibitor. BET protein inhibitors of interest include, but are not limited to, GSK2820151, GSK525762, GS-5829, RO6870810 (IV), BAY1238097, CC-90010, BMS-986158, INCB054329, INCB057643, ODM-207, AZD5153, FT-1101, ABBV-744, ABBV-075, PLX51107, BI894999, OTX015/MK8628, ZEN003694, RVX-000222, CPI-0610, apabetalone and fedratinib.

In some embodiments, the anti-inflammatory agents is an IKK protein inhibitor. In some embodiments, the IKK protein inhibitor is an IKKα, IKKβ, and/or IKKε inhibitor. IKK protein inhibitors of interest include, but are not limited to, B1605906, MLN1120B, PHA-408. LY2409881 PS-145, and BMS-345541.

In some embodiments, the anti-inflammatory agents is an IRAK and/or TLR inhibitor. In some embodiments, the IRAK and/or TLR inhibitor is an IRAK1 inhibitor or IRAK4 inhibitor. IRAK and/or TLR inhibitors of interest include, but are not limited to, BAY1834845, CA-4948, PF-06650833 and pacritinib.

TABLE A

| Compound | Structure |
|---|---|
| GSK525762 | 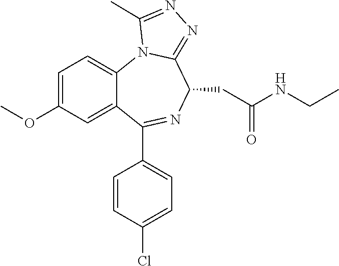 [SMILES: CCNC(=O)C[C@@H]1N=C(c2ccc(Cl)cc2)c2cc(OC)ccc2-n2c(C)nnc21] |
| GS-5829 | 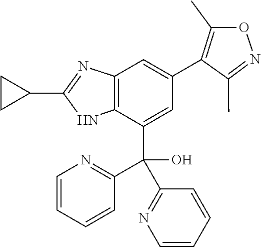 [SMILES: Cc1noc(C)c1-c1cc(C(O)(c2ccccn2)c2ccccn2)c2[nH]c(C3CC3)nc2c1] |
| RO6870810 (IV) | 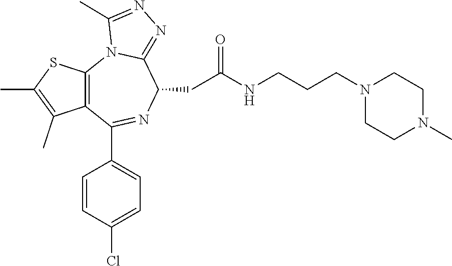 |
| BAY1238097 | 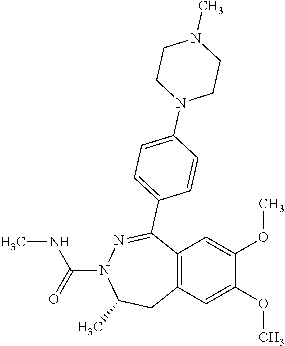 |
| CC-90010 | 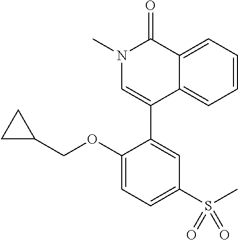 [SMILES: Cn1cc(-c2cc(S(C)(=O)=O)ccc2OCC2CC2)c2ccccc2c1=O] |
| BMS-986158 | 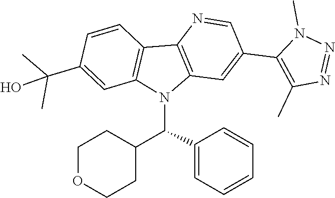 [SMILES: Cc1nnn(C)c1-c1cnc2c3ccc(C(C)(C)O)cc3n([C@H](c3ccccc3)C3CCOCC3)c2c1] |
| INCB054329 | 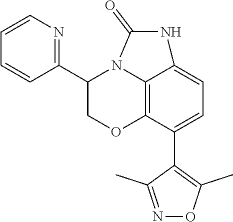 [SMILES: Cc1noc(C)c1-c1ccc2[nH]c(=O)n3c2c1OCC3c1ccccn1] |
| INCB057643 | 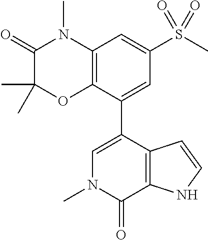 |
| ODM-207 | 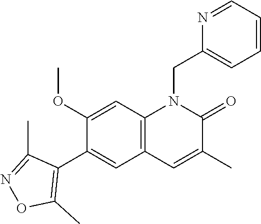 [SMILES: COc1cc2c(cc1-c1c(C)noc1C)cc(C)c(=O)n2Cc1ccccn1] |
| AZD5153 | 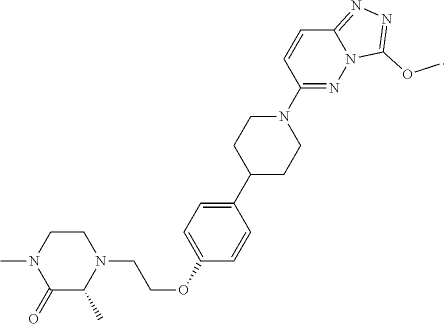 |
| | * |
| | AZD5153 |
| FT-1101 | 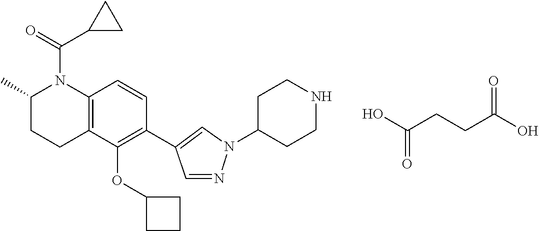 [SMILES: C[C@H]1CCc2c(ccc(-c3cnn(C4CCNCC4)c3)c2OC2CCC2)N1C(=O)C1CC1 ; O=C(O)CCC(=O)O] |
| ABBV-744 | 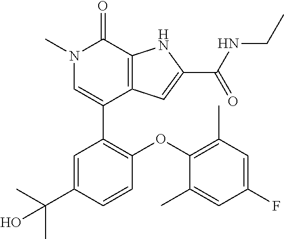 [SMILES: CCNC(=O)c1cc2c(-c3cc(C(C)(C)O)ccc3Oc3c(C)cc(F)cc3C)cn(C)c(=O)c2[nH]1] |
| | ABBV-744 |
| ABBV-075 | 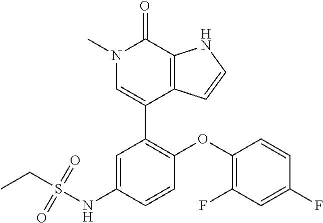 [SMILES: CCS(=O)(=O)Nc1ccc(Oc2ccc(F)cc2F)c(-c2cn(C)c(=O)c3[nH]ccc23)c1] |
| PLX51107 | 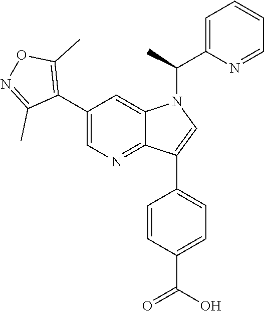 [SMILES: Cc1noc(C)c1-c1cnc2c(-c3ccc(C(=O)O)cc3)cn([C@@H](C)c3ccccn3)c2c1] |
| BI894999 | 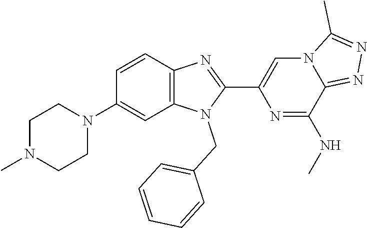 [SMILES: CNc1nc(-c2nc3ccc(N4CCN(C)CC4)cc3n2Cc2ccccc2)cn2c(C)nnc12] |
| OTX015/MK8628 | 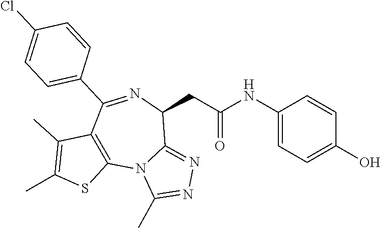 [SMILES: Cc1sc2c(c1C)C(c1ccc(Cl)cc1)=N[C@@H](CC(=O)Nc1ccc(O)cc1)c1nnc(C)n1-2] |
| RVX-000222, | 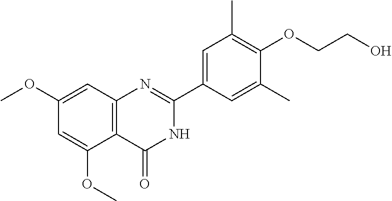 [SMILES: COc1cc(OC)c2c(=O)[nH]c(-c3cc(C)c(OCCO)c(C)c3)nc2c1] |
| CPI-0610 | 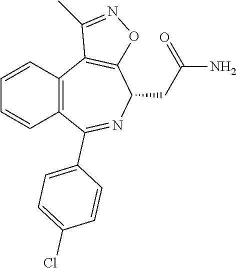 [SMILES: Cc1noc2c1-c1ccccc1C(c1ccc(Cl)cc1)=N[C@H]2CC(N)=O] |
| fedratinib | 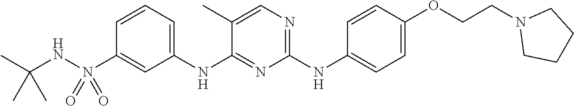 |
| B1605906 | |
| MLN120B | 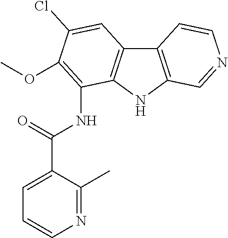 [SMILES: COc1c(Cl)cc2c([nH]c3cnccc32)c1NC(=O)c1cccnc1C] |
| PHA-408 | 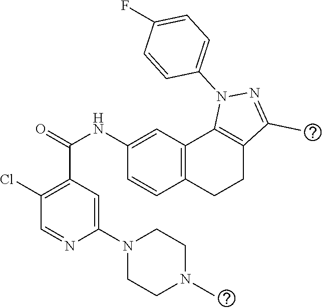 [SMILES: Cc1nn(-c2ccc(F)cc2)c2c1CCc1ccc(NC(=O)c3cc(N4CCN(C)CC4)ncc3Cl)cc1-2] |
| LY2409881 | 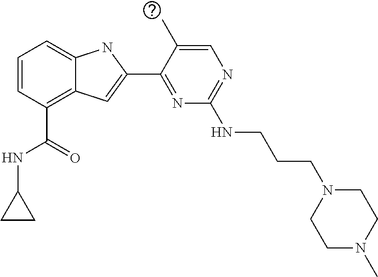 [SMILES: Cc1cnc(NCCCN2CCN(C)CC2)nc1-c1cc2c(C(=O)NC3CC3)cccc2[nH]1] |
| PS-1145 | 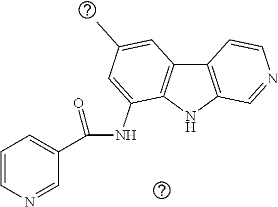 [SMILES: Cc1cc(NC(=O)c2cccnc2)c2[nH]c3cnccc3c2c1] |
| BMS-345541 | 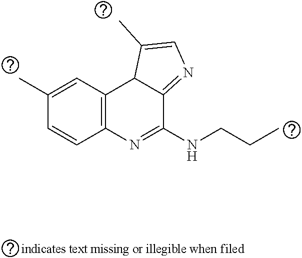 [SMILES: CCCNC1=Nc2ccc(C)cc2C2C(C)=CN=C12] |
| BAY 1834845 | 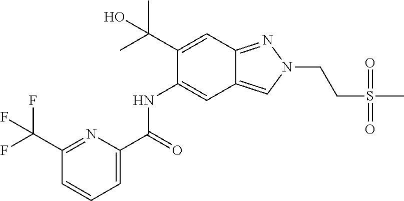 [SMILES: CC(C)(O)c1cc2nn(CCS(C)(=O)=O)cc2cc1NC(=O)c1cccc(C(F)(F)F)n1] |
| CA-4948 | 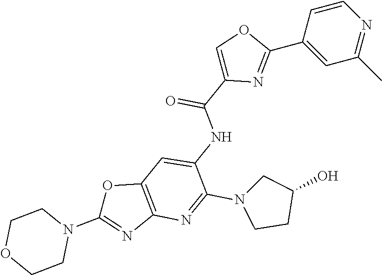 [SMILES: Cc1cc(-c2nc(C(=O)Nc3cc4oc(N5CCOCC5)nc4nc3N3CC[C@@H](O)C3)co2)ccn1] |
| PF-06650833 | 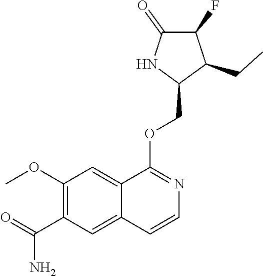 [SMILES: CC[C@@H]1[C@H](F)C(=O)N[C@@H]1COc1nccc2cc(C(N)=O)c(OC)cc12] |
| pacritinib | 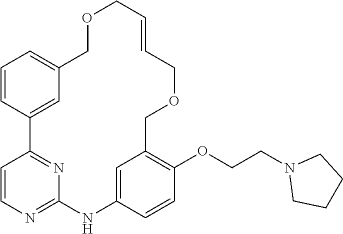 [SMILES: C1=C/COCc2cc(ccc2OCCN2CCCC2)Nc2nccc(n2)-c2cccc(c2)COC/1] |

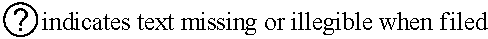

### Momelotinib (MMB)

In one aspect, the present disclosure provides for methods of use of the compound momelotinib (MMB). MMB is sometimes referred to as CYT387. The compound MMB is also identified by the chemical name: N-(cyanomethyl)-4-(2-(4-morpholinophenylamino)pyrimidin-4-yl)benzamide. Salts, including pharmaceutically acceptable salts, solvates, hydrates and/or polymorph forms of MMB can find use in the subject methods disclosed herein.

MMB is a compound that is disclosed in international patent application no. PCT/US2015/035316 and international patent publication no. WO2008/109943, the disclosures of which are herein incorporated by reference. The skilled artisan will find methods that can be used to synthesize MMB in international patent publication no. WO2008/109943.

Table 1 shows the MMB compound structure.

TABLE 1
MMB Structure

| Description | Structure |
|---|---|
| MMB structure | 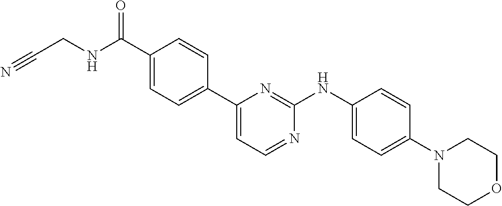 [SMILES: N#CCNC(=O)c1ccc(-c2ccnc(Nc3ccc(N4CCOCC4)cc3)n2)cc1] |

In some cases, a pharmaceutically acceptable salt of MMB is utilized. In some cases, the MMB salt that finds use in the subject methods is a hydrochloride salt. In certain cases, the MMB salt is a dihydrochloride salt. In some cases, the MMB salt is a monohydrochloride salt. In some cases, the MMB salt is a hydrate, such as a monohydrate.

A “solvate” is formed by the interaction of a solvent and a compound. Solvates of salts of the compound described herein are also provided. When the solvent is water, the solvate may be referred to as a hydrate. Hydrates of MMB or MMB salts can also find use in the subject methods.

In some embodiments, the MMB salt is MMB dihydrochloride monohydrate.

In some embodiments, the MMB salt is MMB dihydrochloride anhydrous.

In some embodiments, the MMB or MMB salt composition that is administered is present in a polymorph form, such as a polymorph form that is described in U.S. Pat. No. 9,469,613, the disclosure of which is herein incorporated by reference.

In certain instances, the MMB of the present disclosure may have from 1 to n hydrogen atoms replaced by a deuterium atom (D), in which n is the number of hydrogen atoms in the compound. Such deuterated MMB compounds may increase resistance to metabolism and thus may be useful for increasing the half-life of the compounds described herein when administered to a mammal. See, e.g., Foster, “Deuterium Isotope Effects in Studies of Drug Metabolism,” Trends Pharmacol. Sci., 5(12):524-527 (1984).

### Administration

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Also disclosed herein, the methods of this disclosure include a combination therapy administering an effective amount of MMB and co-administering a second effective amount of a further treatment. Further treatments include, but are not limited to, administering any convenient additional anti-inflammatory agent that finds use in treating a disease or conditions associated with inflammation. In some instances, the additional agent is an NF-kB modulating agent. The term “effective amount” or “therapeutically effective amount” refers to an amount that is effective to ameliorate a symptom of a disease, e.g. as described herein.

Co-administered encompasses methods where MMB and the further treatment are given simultaneously, where MMB and the further treatment are given sequentially, and where either one of, or both of, MMB and the further treatment are given intermittently or continuously, or any combination of: simultaneously, sequentially, intermittently and/or continuously. The skilled artisan will recognize that intermittent administration is not necessarily the same as sequential because intermittent also includes a first administration of an agent and then another administration later in time of that very same agent. Moreover, the skilled artisan understands that intermittent administration also encompasses sequential administration in some aspects because intermittent administration does include interruption of the first administration of an agent with an administration of a different agent before the first agent is administered again. Further, the skilled artisan will also know that continuous administration can be accomplished by a number of routes including i.v. drip or feeding tubes, etc.

Furthermore, and in a more general way, the term “co-administered” encompasses any and all methods where the individual administration of MMB and the individual administration of the further treatment to a subject overlap during any timeframe. In one aspect, MMB and the further treatment are administered on different schedules.

In some aspects, the present disclosure provides for methods where either one of, or both of, or any combination thereof, MMB and/or a further treatment are administered by a route selected from the group consisting of intravenous, subcutaneous, cutaneous, oral, intramuscular, and intraperitoneal. In some aspects, the present disclosure provides for methods where either one of, or both of, or any combination thereof, MMB and/or a further treatment are administered intravenously. In some aspects, the present disclosure provides for methods where either one of, or both of, or any combination thereof, MMB and/or a further treatment are administered orally.

It is understood by the skilled artisan that the unit dose forms of the present disclosure may be administered in the same or different physicals forms, i.e. orally via capsules or tablets and/or by liquid via i.v. infusion, and so on. Moreover, the unit dose forms for each administration may differ by the particular route of administration. Several various dosage forms may exist for either one of, or both of, MMB and a further treatment. Because different medical conditions can warrant different routes of administration, the same components of a combination of MMB and a further treatment described herein may be exactly alike in composition and physical form and yet may need to be given in differing ways and perhaps at differing times to alleviate the condition. For example, a condition such as persistent nausea, especially with vomiting, can make it difficult to use an oral dosage form, and in such a case, it may be necessary to administer another unit dose form, perhaps even one identical to other dosage forms used previously or afterward, with an inhalation, buccal, sublingual, or suppository route instead or as well. The specific dosage form may be a requirement for certain combinations of MMB and a further treatment, as there may be issues with various factors like chemical stability or pharmacokinetics.

In some aspects, the effective amount of MMB and/or the additional agent is less than or equal to the maximum tolerated dose (MTD), less than or equal to the highest non-severely toxic dose (HNSTD), or less than or equal to the No-observed-adverse-effect-level (NOAEL).

In general, the compounds of the present disclosure will be administered in a therapeutically effective amount by any of the accepted modes of administration for agents that serve similar utilities, the actual amount of the compound of the present technology, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, preferably once or twice a day.

An effective amount of such agents can readily be determined by routine experimentation, as can the most effective and convenient route of administration and the most appropriate formulation. Various formulations and drug delivery systems are available in the art. See, e.g., Gennaro. A. R., ed. (1995) Remington's Pharmaceutical Sciences, 18th ed., Mack Publishing Co.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

### EXAMPLES

### Example 1

Momelotinib is a Non-Myelosuppressive, Differentiated JAK Inhibitor with a Pharmacological Profile Amenable to Combinational Therapies.

### Materials.

NF-kB Reporter-HEK293 cell line (BPS Bioscience #60650); MEM medium (Hyclone #SH30024.01); Fetal bovine serum (Life Technologies #10082147); Non-essential amino acid (Corning 25-025-C1); Na-pyruvate (Hyclone #SH30239.01); Pen-strep (Hyclone #SV30010); Hygromycin B (Invitrogen/Thermo-Fisher #10687010); IKK-16 dihydrochloride—NF-kB inhibitor (Sigma SML1138); TNFa (R&D Systems 210-TA); ONE-Step luciferase assay system (BPS Bioscience #60690). Test compounds are from sources listed in Table 2.

TABLE 2
Test compounds used.

| | Compound | Dissolving | Stock | Test Range | Intermediate |
|---|---|---|---|---|---|
| Compound I.D. | Supplied | Solvent | Concentration | (nM) | Dilution |
| MMB/Momelotinib | Powder | DMSO | 10 mM | 0.3-10,000 | 0.1% DMSO in |
| (Selleckchem # S2219, | | | | | assay medium |
| batch # S221901) | | | | | |
| Ruxolitinib (INCB018424) | Powder | DMSO | 10 mM | 0.3-10,000 | 0.1% DMSO in |
| (Chemietek # CT-INCB, lot | | | | | assay medium |
| # 17) | | | | | |
| Fedratinib (TG101348) | Powder | DMSO | 10 mM | 0.3-10,000 | 0.1% DMSO in |
| (Selleckchem # S2736, lot # | | | | | assay medium |
| S273603) | | | | | |
| Pacritinib (SB1518) | Powder | DMSO | 10 mM | 0.3-10,000 | 0.1% DMSO in |
| (Selleckchem # S8057, lot # | | | | | assay medium |
| S805701) | | | | | |
| CPI-0610 | Powder | DMSO | 10 mM | 0.3-10,000 | 0.1% DMSO in |
| (BioVision # B1201-5, lot | | | | | assay medium |
| #3F08B12010) | | | | | |
| *IKK-16 dihydrochloride | Powder | DMSO | 10 mM | 1.95-1,000 | 0.1% DMSO in |
| | | | | | assay medium |

*Reference compound

### Methods.

Cell culture. Cells were cultured in MEM medium with 10% FBS, 1% non-essential amino acid, 1 mM Na-pyruvate, 1% Pen-strep, and 50 μg/mL Hygromycin B.

### Assay Conditions.

To perform the NF-kB luciferase reporter assay, NF-kB Reporter—HEK293 cells were seeded at 30,000 cells per well into white clear-bottom 96-well microplate in 60 μL of assay medium (growth medium without Hygromycin B). Cells were incubated at 37° C. and 5% CO₂ for 5 hours to allow them attach. A serial dilution of compounds was prepared in assay medium without Hygromycin B. Thirty μL of diluted compounds was added to the treatment wells at the end of the day. Thirty μL of assay medium with the same concentration of DMSO without compound was added to the control wells. One hundred μL of assay medium with DMSO was added to cell-free control wells for determining background luminescence. Cells were incubated at 37° C. and 5% CO₂ overnight.

The next day ten μL of diluted human TNFa in assay medium was added to wells (final [TNFa]=10 ng/mL). Ten μL of assay medium was added to unstimulated control wells. Cells were incubated at 37° C. and 5% CO₂ for 5-6 hours. After treatment, cells were lysed and the luciferase assay was performed using ONE-Step luciferase assay system: add 100 μL of One-Step Luciferase reagent per well and rock at room temperature for ˜30 minutes. Luminescence was measured using a luminometer (BioTek Synergy™ 2 microplate reader).

### Results.

TABLE 3
The IC₅₀ values of the compounds against NF-kB reporter activity.

| | | IC₅₀ (uM) | |
|---|---|---|---|
| | | Lock top and | Percent |
| | | bottom of | Inhibition |
| Compounds | IC₅₀ (uM) | curve | at 10 uM |
| MMB/Momelotinib | 0.47 | 0.43 | 100 |
| Ruxolitinib (INCB018424) | not determined | not determined | −11 |
| Fedratinib (TG101348) | 6.1 | 2.9 | 99 |
| Pacritinib (SB1518) | 1.4 | 1.2 | 100 |
| CPI-0610 | 0.27 | 0.54 | 78 |
| *IKK-16 dihydrochloride | 0.33 | 0.26 | 88 |

*Reference compound

Cellular data shows MMB downregulates NF-κB signaling in a luciferase assay (whereas ruxolitinib (RUX), ran in parallel, did not). This experiment was performed to compare the effects of MMB on NF-κB signaling with those of the JAK inhibitors listed in the table above on NF-κB signaling.

Conclusions: These novel data suggest MMB harbors unique NF-κB activity in addition to JAK1, JAK2 and ACVR1 inhibitory activity.

### EMBODIMENTS OF THE DISCLOSURE

1. A method of treating an inflammatory disease or disorder, the method comprising: administering to a subject in need thereof a therapeutically effective amount of momelotinib (MMB) or a pharmaceutically acceptable salt thereof; and administering to the subject one or more anti-inflammatory agents.
2. The method of embodiment 1, wherein the one or more anti-inflammatory agents is an agent that modulates NF-κB activity.
3. The method of embodiment 1 or 2, wherein the one or more anti-inflammatory agents is a BET protein inhibitor.
4. The method of embodiment 3, wherein the BET protein inhibitor is a BRD4 inhibitor.
5. The method of embodiment 3 or 4, wherein the BET protein inhibitor is selected from GSK2820151, GSK525762, GS-5829, RO6870810 (IV), BAY1238097, CC-90010. BMS-986158, INCB054329, INCB057643, ODM-207, AZD5153, FT-1101, ABBV-744, ABBV-075, PLX51107, BI894999, OTX015/MK8628, ZEN003694, RVX-000222, CPI-0610, apabetalone and fedratinib.
6. The method of embodiment 1 or 2, wherein the one or more anti-inflammatory agents is an IKK protein inhibitor.
7. The method of embodiment 6, wherein the IKK inhibitor is an IKKα, IKKβ, and/or IKKε inhibitor.
8. The method of embodiment 6 or 7, wherein the IKK inhibitor is selected from B1605906, MLN120B, PHA-408, LY2409881. PS-1145, and BMS-345541.
9. The method of embodiment 1 or 2, wherein the one or more anti-inflammatory agents is an IRAK and/or TLR inhibitor
10. The method of embodiment 9, wherein the IRAK and/or TLR inhibitor is an IRAK1 inhibitor or IRAK4 inhibitor.
11. The method of embodiment 9 or 10, wherein the IRAK and/or TLR inhibitor is selected from BAY1 834845, CA-4948, PF-06650833 and pacritinib.
12. The method of embodiment 2, wherein the one or more anti-inflammatory agents is a protein or small molecule agent.
13. The method of any one of embodiments 1-12, wherein treating the inflammatory disease or disorder comprises at least ameliorating one or more symptoms of the disease or disorder.
14. The method of embodiment 13, wherein amelioration of the one or more symptoms is increased as compared to a monotherapy with momelotinib (MMB) or a monotherapy with the one or more anti-inflammatory agents alone.
15. The method of any one of embodiments 1-14, wherein the administering results in reduction in NF-κB pathway activity and modulation of JAK-STAT and ACVR1/SMAD signaling in cells of the subject.
16. The method of any one of embodiments 1-15, wherein the subject has a chronic inflammatory disease, an autoimmune disease or cancer.
17. The method of any one of embodiments 1-16, wherein the MMB or a pharmaceutically acceptable salt thereof is administered orally.
18. The method of any one of embodiments 1-17, wherein the subject is human.
19. A method of treating cancer comprising: administering to a subject in need thereof a therapeutically effective amount of momelotinib (MMB) or a pharmaceutically acceptable salt thereof; and administering to the subject one or more anti-inflammatory agents.
20. The method of embodiment 19, wherein the one or more anti-inflammatory agents is an agent that modulates NF-κB activity.
21. The method of embodiment 19 or 20, wherein the one or more anti-inflammatory agents is a BET protein inhibitor.
22. The method of embodiment 21, wherein the BET protein inhibitor is a BRD4 inhibitor.
23. The method of embodiment 21 or 22, wherein the BET protein inhibitor is selected from GSK2820151, GSK525762, GS-5829, RO6870810 (IV), BAY1238097, CC-90010, BMS-986158, INCB054329, INCB057643, ODM-207, AZD5153, FT-1101, ABBV-744, ABBV-075, PLX51107, BI894999, OTX015/MK8628, ZEN003694, RVX-000222, CPI-0610, apabetalone and fedratinib.
24. The method of embodiment 19 or 20, wherein the one or more anti-inflammatory agents is an IKK protein inhibitor.
25. The method of embodiment 24, wherein the IKK inhibitor is an IKKα, IKKβ, and/or IKKε inhibitor.
26. The method of embodiment 24 or 25, wherein the IKK inhibitor is selected from B1605906, MLN120B, PHA-408, LY2409881, PS-1145, and BMS-345541.
27. The method of embodiment 19 or 20, wherein the one or more anti-inflammatory agents is an IRAK and/or TLR inhibitor
28. The method of embodiment 27, wherein the IRAK and/or TLR inhibitor is an IRAK1 inhibitor or IRAK4 inhibitor.
29. The method of embodiment 27 or 28, wherein the IRAK and/or TLR inhibitor is selected from BAY1 834845, CA-4948, PF-06650833 and pacritinib.
30. The method of embodiment 20, wherein the one or more anti-inflammatory agents is a protein or small molecule agent.
31. The method of any one of embodiments 19-30, wherein treating cancer comprises at least ameliorating one or more symptoms.
32. The method of embodiment 31, wherein amelioration of the one or more symptoms is increased as compared to a monotherapy with momelotinib (MMB) or a monotherapy with the one or more anti-inflammatory agents alone.
33. The method of any one of embodiments 19-32, wherein the subject has myelofibrosis.
34. The method of embodiment 33, wherein the anti-inflammatory agent is CPI-0610.
35. The method of any one of embodiments 19-34, wherein the administering results in reduction in NF-κB pathway activity and modulation of JAK-STAT and ACVR1/SMAD signaling in cells of the subject.
36. The method of any one of embodiments 19-35, wherein the MMB or a pharmaceutically acceptable salt thereof is administered orally.
37. The method of any one of embodiments 19-36, wherein the subject is human.

1. A method of treating an inflammatory disease or disorder, the method comprising:
administering to a subject in need thereof a therapeutically effective amount of momelotinib (MMB) or a pharmaceutically acceptable salt thereof; and administering to the subject one or more anti-inflammatory agents.

2. The method of claim 1, wherein the one or more anti-inflammatory agents is an agent that modulates NF-κB activity.

3. The method of claim 1, wherein the one or more anti-inflammatory agents is a BET protein inhibitor; optionally wherein the BET protein inhibitor is a BRD4 inhibitor.

4. The method of claim 3, wherein the BET protein inhibitor is selected from GSK2820151, GSK525762, GS-5829, RO6870810 (IV), BAY1238097, CC-90010, BMS-986158, INCB054329, INCB057643, ODM-207, AZD5153, FT-1101, ABBV-744, ABBV-075, PLX51107, BI894999, OTX015/MK8628, ZEN003694, RVX-000222, CPI-0610, apabetalone and fedratinib.

5. The method of claim 1, wherein the one or more anti-inflammatory agents is an IKK protein inhibitor; optionally wherein the IKK inhibitor is an IKKα, IKKβ, and/or IKKε inhibitor.

6. The method of claim 4, wherein the IKK inhibitor is selected from B1605906, MLN120B, PHA-408, LY2409881, PS-1145, and BMS-345541.

7. The method of claim 1, wherein the one or more anti-inflammatory agents is an IRAK and/or TLR inhibitor; optionally wherein the IRAK and/or TLR inhibitor is an IRAK1 inhibitor or IRAK4 inhibitor.

8. The method of claim 7, wherein the IRAK and/or TLR inhibitor is selected from BAY1 834845, CA-4948, PF-06650833 and pacritinib.

9. The method of claim 1, wherein treating the inflammatory disease or disorder comprises at least ameliorating one or more symptoms of the disease or disorder; optionally wherein amelioration of the one or more symptoms is increased as compared to a monotherapy with momelotinib (MMB) or a monotherapy with the one or more anti-inflammatory agents alone.

10. The method of claim 1, wherein the administering results in reduction in NF-κB pathway activity and modulation of JAK-STAT and ACVR1/SMAD signaling in cells of the subject.

11. The method of claim 1, wherein the subject has a chronic inflammatory disease, an autoimmune disease or cancer.

12. A method of treating cancer comprising:
administering to a subject in need thereof a therapeutically effective amount of momelotinib (MMB) or a pharmaceutically acceptable salt thereof; and administering to the subject one or more anti-inflammatory agents.

13. The method of claim 12, wherein the one or more anti-inflammatory agents is an agent that modulates NF-κB activity.

14. The method of claim 12, wherein the one or more anti-inflammatory agents is a BET protein inhibitor; optionally wherein the BET protein inhibitor is a BRD4 inhibitor.

15. The method of claim 14, wherein the BET protein inhibitor is selected from GSK2820151, GSK525762, GS-5829, RO6870810 (IV), BAY1238097, CC-90010, BMS-986158, INCB054329, INCB057643, ODM-207, AZD5153, FT-1101, ABBV-744, ABBV-075, PLX51107, BI894999, OTX015/MK8628, ZEN003694, RVX-000222, CPI-0610, apabetalone and fedratinib.

16. The method of claim 12, wherein the one or more anti-inflammatory agents is an IKK protein inhibitor; optionally wherein the IKK inhibitor is an IKKα, IKKβ, and/or IKKε inhibitor.

17. The method of claim 16, wherein the IKK inhibitor is selected from B1605906, MLN120B, PHA-408, LY2409881, PS-1145, and BMS-345541.

18. The method of claim 12, wherein the one or more anti-inflammatory agents is an IRAK and/or TLR inhibitor.

19. The method of claim 18, wherein the IRAK and/or TLR inhibitor is an IRAK1 inhibitor or IRAK4 inhibitor; optionally wherein the IRAK and/or TLR inhibitor is selected from BAY1 834845, CA-4948, PF-06650833 and pacritinib.

20. The method of claim 12, wherein treating cancer comprises at least ameliorating one or more symptoms; optionally wherein amelioration of the one or more symptoms is increased as compared to a monotherapy with momelotinib (MMB) or a monotherapy with the one or more anti-inflammatory agents alone.

21. The method of claim 12, wherein the subject has myelofibrosis; optionally wherein the anti-inflammatory agent is CPI-0610.

22. The method of claim 12, wherein the administering results in reduction in NF-κB pathway activity and modulation of JAK-STAT and ACVR1/SMAD signaling in cells of the subject.
